(19) United States
(12) Patent Application Publication
(10) Pub. No.: US 2008/0125749 A1
(43) Pub. Date: May 29, 2008

(54) SELF-POWERED MEDICAL DEVICES

(75) Inventor: Gregory Olson, Elk River, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 11/468,249

(22) Filed: Aug. 29, 2006

(51) Int. Cl. A61M 25/18

(52) U.S. Cl. 604/523

(57) ABSTRACT

Medical devices, including intravascular medical devices, may be constructed to include on-board power generation capabilities adapted to provide power to an electrical load, such as a therapeutic element, that is disposed upon or formed within the medical device. An electrical power generator may be disposed upon or formed within a proximal portion of a medical device.

### TECHNICAL FIELD

The invention relates generally to medical devices and more particularly to medical devices that include electrical power.

### BACKGROUND

Some medical and/or therapeutic treatments, including intravascular treatments, involve the use of therapeutic elements that require electrical power. Some therapeutic elements have been powered via batteries, AC current, or some combination thereof. A need remains for improved techniques for powering medical devices, especially intravascular medical devices.

### SUMMARY

The present invention pertains generally to improved techniques for powering medical devices such as intravascular medical devices. The present invention pertains to medical devices employing improved power generation capability.

Accordingly, an illustrative but non-limiting example of the invention may be found in a medical device that includes a proximal hub and an elongate shaft that extends distally from the proximal hub. An electrical load may be disposed within a distal region of the elongate shaft. An electrical generator disposed within the proximal hub may be in electrical communication with the electrical load.

Another illustrative but non-limiting example of the invention may be found in a medical device hub. The medical device hub includes a hub body and a lumen extending through the hub body. An induction coil and a magnet that is movable with respect to the induction coil may be both disposed within the hub body. The medical device hub may also include a user-actuated apparatus that is adapted to move the magnet relative to the induction coil.

The above summary of the present invention is not intended to describe each disclosed embodiment or every implementation of the present invention. The Figures, Detailed Description and Examples which follow more particularly exemplify these embodiments.

### BRIEF DESCRIPTION OF THE FIGURES

The invention may be more completely understood in consideration of the following detailed description of various embodiments of the invention in connection with the accompanying drawings, in which:

FIG. 1 is a side elevation view of a catheter in accordance with an illustrative but non-limiting example of the invention;

FIG. 2 is a side elevation view of a medical device hub in accordance with an illustrative but non-limiting example of the invention;

FIG. 3 is a cross-section taken along line 3-3 of FIG. 2;

FIG. 4 is a top view of a medical device hub in accordance with an illustrative but non-limiting example of the invention;

FIG. 5 is a partial cross-section taken along line 5-5 of FIG. 4;

FIG. 6 is a top view of a medical device hub in accordance with an illustrative but non-limiting example of the invention;

FIG. 7 is a partial cross-section taken along line 5-5 of FIG. 6; and

FIG. 8 is a top view of a medical device hub in accordance with an illustrative but non-limiting example of the invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention. The Figures are not drawn to any particular scale and are simply presented for ease of illustration.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The drawings, which are not necessarily to scale, depict illustrative embodiments of the claimed invention.

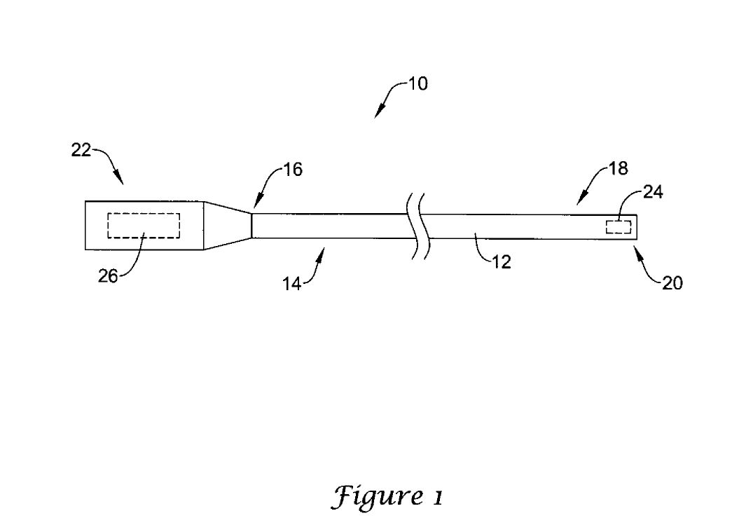

FIG. 1 is a plan view of catheter 10 in accordance with an illustrative but non-limiting example of the present invention. The catheter 10 can be any of a variety of different catheters and may, in some instances, include both an electrical load and an electrical generator, as will be discussed in greater detail hereinafter.

In some instances, the catheter 10 can be an intravascular catheter. Examples of intravascular catheters include balloon catheters, atherectomy catheters, drug delivery catheters, stent delivery catheters, diagnostic catheters and guide catheters. The intravascular catheter 10 can be sized in accordance with its intended use. The catheter 10 can, for example, have a length that is in the range of about 100 to 150 centimeters and can have any useful diameter. Except as discussed herein, the intravascular catheter 10 can be manufactured using conventional techniques.

In the illustrated embodiment, the intravascular catheter 10 includes an elongate shaft 12 that has a proximal region 14 defining a proximal end 16 and a distal region 18 defining a distal end 20. A proximal hub 22 can be connected to the proximal end 16 of the elongate shaft 12. The proximal hub 22 can be of conventional design, other than as discussed herein and can be attached using conventional techniques. It is also recognized that alternative hub designs can be incorporated into embodiments of the present invention.

The elongate shaft 12 can include one or more shaft segments having varying degrees of flexibility. For example, the elongate shaft may include a relatively stiff proximal portion, a relatively flexible distal portion and an intermediate position disposed between the proximal and distal portions having a flexibility that is intermediate to both.

In some cases, the elongate shaft 12 may be formed of a single polymeric layer. In some instances, the elongate shaft 12 may include an inner liner such as an inner lubricious layer and an outer layer. In some cases, the elongate shaft 12 may include a reinforcing braid layer disposed between the inner and outer layers. The elongate shaft 12 is considered herein as generically representing a catheter to which various elements can be added to provide the catheter 10 with adjustable stiffness.

If the elongate shaft 12 includes an inner liner, the inner liner can include or be formed from a coating of a material having a suitably low coefficient of friction. Examples of suitable materials include perfluoro polymers such as polytetrafluoroethylene (PTFE), better known as TEFLON®, high density polyethylene (HDPE), polyarylene oxides, polyvinylpyrolidones, polyvinylalcohols, hydroxy alkyl cellulosics, algins, saccharides, caprolactones, and the like, and mixtures and combinations thereof.

The elongate shaft 12 can include, as an outer layer or layers, any suitable polymer that will provide the desired strength, flexibility or other desired characteristics. Polymers with low durometer or hardness can provide increased flexibility, while polymers with high durometer or hardness can provide increased stiffness. In some embodiments, the polymer material used is a thermoplastic polymer material. Some examples of suitable materials include polyurethane, elastomeric polyamides, block polyamide/ethers (such as PEBAX®), silicones, and co-polymers. The outer polymer layer 32 can be a single polymer, multiple longitudinal sections or layers, or a blend of polymers. In some instances, a thermoplastic polymer such as a co-polyester thermoplastic elastomer, for example, available commercially under the ARNITEL® name, can be used.

In some instances, as noted above, the illustrative intravascular catheter 10 may include an electrical load 24, which is shown diagrammatically in phantom within the distal region 18 of the elongate shaft 12. The electrical load 24 may represent any diagnostic, therapeutic, or other device or apparatus that may be employed within a catheter and that may require electrical energy. In some instances, the electrical load 24 may represent a therapeutic device such as a thermal device or a sampling device. The electrical load 24 may represent or include a light. Lights are employed within some catheters for illumination.

In some instances, the electrical load 24 may represent an electrorheological fluid that can be gelled by applying a voltage across the fluid. Electrorheological fluids are described in greater detail in U.S. Ser. No. 11/190,983, filed Jul. 27, 2005, entitled MEDICAL DEVICES WITH VARIABLE STIFFNESS, which application is incorporated by reference herein.

In some cases, as noted above, the intravascular catheter 10 may include an electrical generator 26. In FIG. 1, the electrical generator 26 is diagrammatically shown in phantom within the proximal hub 22, but the electrical generator 26 may also be disposed exterior to but proximate the proximal hub 22, if desired. The electrical generator 26 may take any suitable form, provided it can be user-actuated, and can provide sufficient power to operate the electrical load 24. Examples of suitable electrical generators will be discussed with respect to subsequent Figures.

In some cases, the electrical generator 26 may be adapted such that it can be activated and/or manipulated by a user to provide power while the intravascular catheter 10 is deployed within a patient. In some instances, the electrical generator 26 may be adapted to permit power generation and storage prior to deployment. If desired, for example, the electrical generator 26 could be electrically connected to a capacitor that may permit storage of the power generated. In some cases, the electrical generator 26 may be in electrical communication with circuitry that may be used, for example, to control and/or adjust the level of power generation.

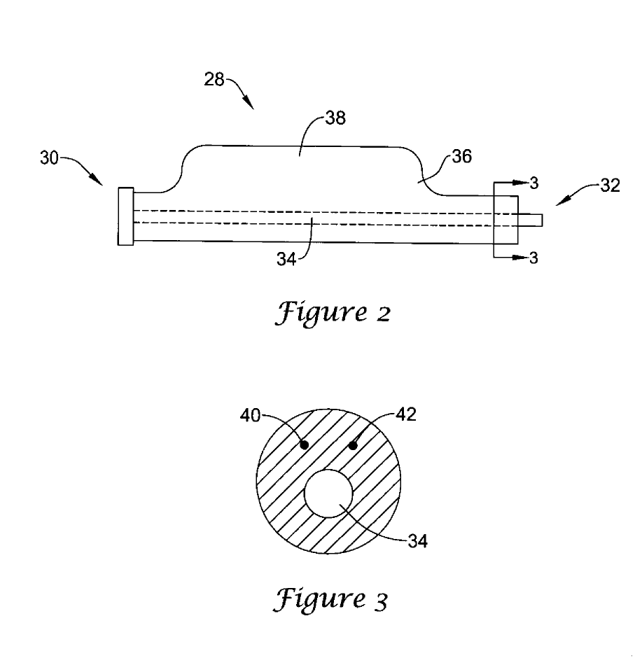

FIG. 2 is a diagrammatic view of an illustrative but non-limiting medical device hub 28 including on-board power generation. The medical device hub 28 includes a proximal end 30, a distal end 32 and a lumen 34 (shown in phantom) extending between the proximal end 30 and the distal end 32. The distal end 32 may be adapted to be connected, such as via a luer fitting, to any appropriate diagnostic or therapeutic catheter. The proximal end 30 may be adapted to permit access to the lumen 34.

The medical device hub 28 includes a hub body 36, through which the lumen 34 extends. The hub body 36 also has a power generation section 38. While specific examples of electrical generators will be discussed with respect to subsequent Figures, it should be noted that the power generation section 38 generically shown here includes both apparatus for generating power as well as structure by which a user may activate and/or manipulate the power generation apparatus.

FIG. 3 is a cross-section taken through the medical device hub 28, near its distal end 32. It can be seen that the medical device hub 28 includes a first electrical contact 40 and a second electrical contact 42. The first electrical contact 40 and the second electrical contact 42 may extend distally from the power generation section 38, and may be configured to provide electrical contact with a device that may be secured to the distal end 32 of the medical device hub 28.

The first electrical contact 40 and the second electrical contact 42 may be molded into the medical device hub 28. The first electrical contact 40 and the second electrical contact 42 may be formed of any suitable material. In some cases, the first and second electrical contact 40 and 42 may include or be formed of copper wires. In some cases, it is contemplated that the first and/or second electrical contacts 40 and 42 may, if desired, extend exterior to the medical device hub 28 between the power generation section 38 and the distal end 32.

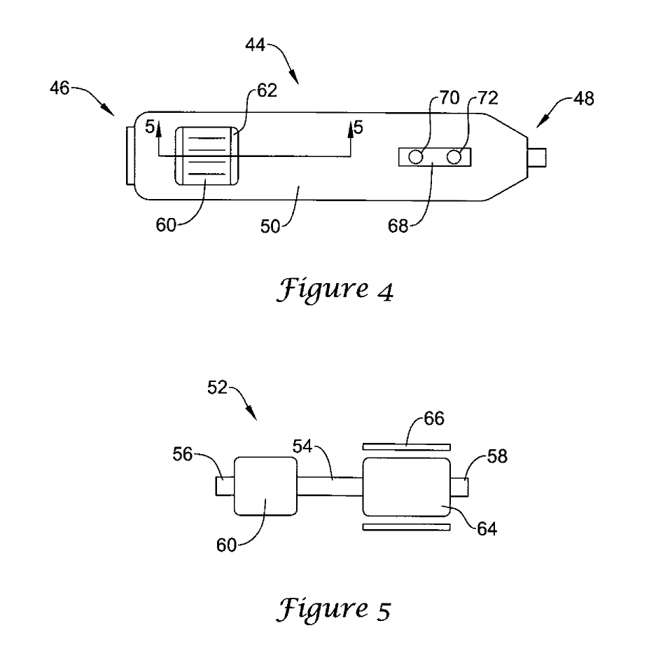

FIG. 4 is a top view of an illustrative but non-limiting medical device hub 44 having a proximal end 46, a distal end 48 and a hub body 50. While not shown in this view, a lumen may extend through the hub body 50 between the proximal end 46 and the distal end 48. In some instances, the medical device hub 44 may not include any lumens. Rather, a more traditional hub (such as proximal hub 22 discussed previously) may be secured to the distal end 48, thereby providing device and/or fluid access to any lumens within a catheter or similar device deployed distal of the traditional hub.

In some cases, the medical device hub 44 does include one or more lumens that may be used for any suitable purpose such as advancing a wire or other device, or providing a fluid such as a contrast fluid. In order to accommodate the one or more lumens, and as seen in the power generation section 38 of FIG. 2, the medical device hub 44 may include a raised or enlarged section accommodating the power generation apparatus. Such a raised or enlarged section would not be visible, of course, in the top view shown in FIG. 4.

FIGS. 4 and 5 illustrate a power generation apparatus 52 that is positioned within the medical device hub 44. Power generation apparatus 52 includes a shaft 54 that may be accommodated within the hub body 50. In some cases, the hub body 50 may, for example, include one or more bearings that are configured to support the shaft 54 while permitting the shaft 54 to rotate. The shaft 54 has a proximal end 56 and a distal end 58.

A thumb wheel 60 is secured to the shaft 54 near the proximal end 56 thereof. As can be seen in FIG. 4, the thumb wheel 60 can be accessed from exterior to the hub body 50 via a thumb wheel aperture 62. It can be seen that a physician or other healthcare professional may rotate the shaft 54 simply by rotating the thumb wheel 60.

Nearer the distal end 58, a rotor 64 is secured onto the shaft 54. A stator 66 is positioned such that the rotor 64 may rotate within the stator 66 when the shaft 54 is rotated by manipulating the thumb wheel 60. One of the rotor 64 and the stator 66 may include or be formed from a magnet, while the other of the rotor 64 and the stator 66 may include or be formed of a wire coil. In some instances, the rotor 64 is a magnet and the stator 66 is a wire coil. Rotating the magnet relative to the wire coil can cause an inductive current within the wire col. This current may be provided directly to an electrical load 24 (FIG. 1), or can be stored prior to use.

In some instances, as illustrated, the medical device hub 44 may include a control board 68. If desired, the control board 68 may include an on/off switch 70 that can be used to provide or interrupt power from the power generation apparatus 52. As shown, the on/off switch 70 is a push button, but other switch types are known and may be appropriate.

The control board 68 may, if desired, also include a charge indicator light 72. The charge indicator light 72, if present, may indicate whether or not a sufficient level of power generation has been reached. For example, the charge indicator light 72 may remain unlit until a threshold power level has been reached, and may light once this threshold power level has been reached and/or exceeded. The charge indicator light 72 may include any suitable illumination source. In some cases, the charge indicator light 72 may be a light emitting diode (LED). While not illustrated, the control board 68 could also include a second light indicating, for example, excessive power generation.

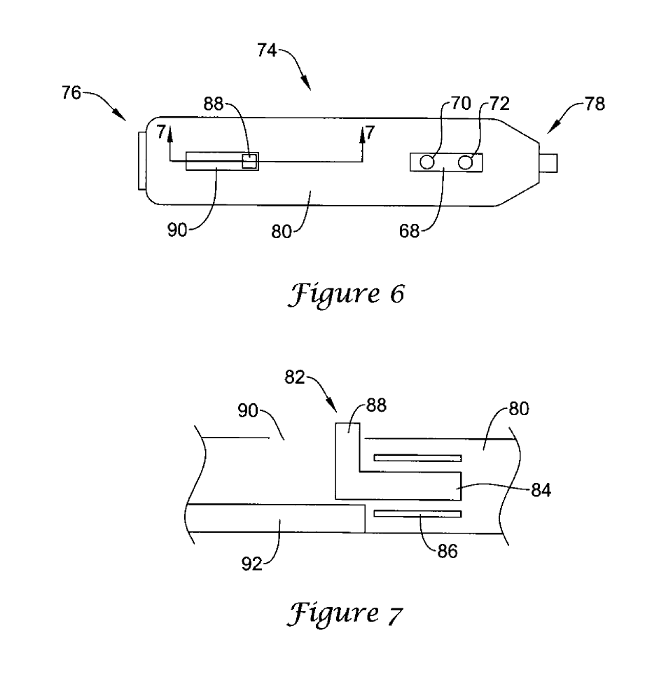

FIG. 6 is a top view of an illustrative but non-limiting medical device hub 74 having a proximal end 76, a distal end 78 and a hub body 80. The medical device hub 74 may, if desired, include the control board 68 discussed above. While not shown in this view, a lumen may extend through the hub body 80. In some cases, a more traditional hub (such as proximal hub 22 discussed previously) may be secured to the distal end 78.

FIGS. 6 and 7 illustrate a power generation apparatus 82 that is positioned within the medical device hub 74. The power generation apparatus 82 employs sliding, or relative axial movement between a rotor and a stator, rather than the rotational motion showed, for example, in FIGS. 4 and 5. In particular, the power generation apparatus 82 includes a rotor 84 and a stator 86. In some instances, the rotor 84 may be a magnet while the stator 86 may be a wire coil. Sliding the magnet relative to the wire coil can cause an inductive current within the wire col. This current may be provided directly to an electrical load 24 (FIG. 1), or can be stored prior to use.

The rotor 84 includes an extension 88 that extends radially outwardly from the rotor 84 and may be configured to function as a handle or tab that may be manipulated from a position exterior to the hub body 80. The extension 88 can be seen as extending through an elongate opening 90. The elongate opening 90 may be sized to permit the extension 88 to move sufficiently far back and forth to permit the rotor 84 to move between a position in which the rotor 84 is disposed within the stator 86 and a position in which the rotor 84 is disposed exterior to the stator 86. The extension 88 may be integrally formed with the rotor 84, or the extension 88 may be separately formed and then subsequently secured to the rotor 84.

In some instances, the hub body 80 may include a support structure 92 that is sized and configured to support the rotor 84 while permitting the rotor 84 to slide back and forth. The support structure 92 may be integrally molded with the hub body 80, or may be separately formed and then subsequently inserted into the hub body 80. In some cases, the support structure 92 may include or be coated with a low friction material such as polyethylene or even polytetrafluoroethylene (better known as TEFLON®).

The power generation apparatus 82 generates power by sliding the rotor 84 back and forth through the stator 86. As illustrated, the rotor 84 is moved back and forth by moving the extension 88 back and forth. It is contemplated, however, that this axial movement could also be achieved by connecting the rotor 84 to a cammed lever (not shown). The lever could be angled back and forth about a pivot point, and a cam could be used to convert this movement into linear motion.

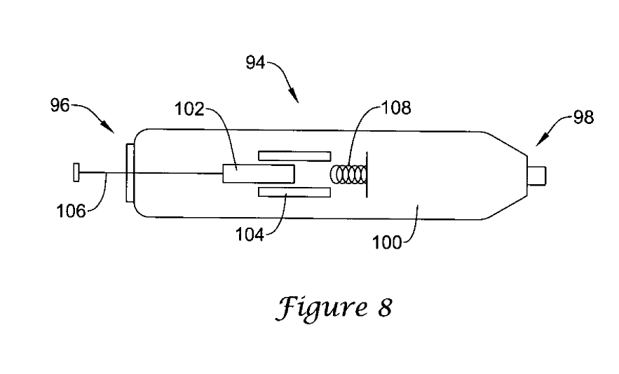

Another technique for achieving relative axial movement between a rotor and a stator is shown in FIG. 8. FIG. 8 is a partial cross-section top view of an illustrative but non-limiting medical device hub 94. The medical device hub 94 has a proximal end 96, a distal end 98 and a hub body 100. A rotor 102 is slidingly disposed relative to a stator 104. In some instances, the rotor 102 may be a magnet while the stator 104 may be a wire coil. Sliding the magnet relative to the wire coil can cause an inductive current within the wire col. This current may be provided directly to an electrical load 24 (FIG. 1), or can be stored prior to use.

A plunger 106 extends proximally from the rotor 102 to a position exterior the hub body 100. A spring assembly 108 is positioned near a distal end of travel that the rotor 102 is permitted. While a spring is illustrated, the spring assembly 108 could include or be formed from a suitably elastomeric material. Pushing the plunger 106 distally causes the rotor 102 to slide into the stator 104. The rotor 102 will contact the spring assembly 108 and be pushed back through and out of the stator 104. Thus, the medical device hub 94 may be considered as employing a spring-loaded plunger.

The devices described herein may include a variety of different materials. These materials may include metals, metal alloys, polymers, metal-polymer composite, and the like, or any other suitable material. Some examples of suitable metals and metal alloys include stainless steel, such as 304V, 304L, and 316LV stainless steel; mild steel; nickel-titanium alloy such as linear-elastic or super-elastic nitinol, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), hastelloy, monel 400, inconel 825, or the like; other Co—Cr alloys; platinum enriched stainless steel; or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN®) available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

In addition, the devices described herein may also be doped with or otherwise include a radiopaque material. Radiopaque materials are understood to be materials capable of producing a relatively bright image on a fluoroscopy screen or another imaging technique during a medical procedure. This relatively bright image aids the user of filtering device in determining their location. Some examples of radiopaque materials can include, but are not limited to, gold, platinum, molybdenum, palladium, tantalum, tungsten or tungsten alloy, plastic material loaded with a radiopaque filler, and the like.

It should be understood that this disclosure is, in many respects, only illustrative. Changes may be made in details without exceeding the scope of the invention. The invention's scope is, of course, defined in the language in which the appended claims are expressed.

We claim:

1. An medical device comprising:
a proximal hub;
an elongate shaft extending distally from the proximal hub, the elongate shaft having a distal region;
an electrical load disposed within the distal region of the elongate shaft; and
an electrical generator disposed within the proximal hub, the electrical generator in electrical communication with the electrical load.

2. The medical device of claim 1, wherein the electrical generator comprises a magnet and a coil, one of the magnet and the coil movable relative to another of the magnet and the coil.

3. The medical device of claim 2, wherein the proximal hub further comprises a user-actuatable apparatus adapted to move one of the magnet and the coil relative to another of the magnet and the coil.

4. The medical device of claim 3, wherein the user-actuatable apparatus is configured to move the magnet relative to the coil.

5. The medical device of claim 4, wherein the user-actuatable apparatus is adapted to provide rotary movement to the magnet.

6. The medical device of claim 4, wherein the user-actuatable apparatus is adapted to provide linear movement to the magnet.

7. The medical device of claim 1, wherein the user-actuatable apparatus comprises a thumb wheel.

8. The medical device of claim 1, wherein the user-actuatable apparatus comprises a sliding element.

9. The medical device of claim 1, wherein the user-actuatable comprises a spring-loaded plunger.

10. The medical device of claim 1, wherein the electrical generator is adapted to generate electrical power during deployment of the medical device.

11. The medical device of claim 1, wherein the electrical generator is adapted to generate and store electrical power prior to deployment of the medical device.

12. The medical device of claim 1, wherein the proximal hub further comprises a capacitor for storing electrical power generated prior to deployment.

13. The medical device of claim 1, wherein the electrical load comprises a therapeutic element adapted for activation via power generated by the electrical generator.

14. The medical device of claim 13, wherein the therapeutic element comprises one of an electrorheological fluid disposed between electrical contacts, a diagnostic sensor, a moving element or a thermal element.

15. A medical device hub, comprising:
a hub body having a distal end, a proximal end and a lumen extending therebetween;
an induction coil and a magnet movable with respect to the induction coil, the induction coil and magnet disposed within the hub body; and
a user-actuated apparatus adapted to move the magnet relative to the induction coil.

16. The medical device hub of claim 15, wherein the distal end is adapted for releasable securement to an elongate medical device.

17. The medical device hub of claim 15, wherein the distal end comprises electrical contacts for providing electrical communication with an elongate medical device comprising an electrical load.

18. The medical device hub of claim 15, wherein the proximal end is adapted to provide access to the lumen extending through the hub body.

19. The medical device hub of claim 15, further comprising an on/off switch.

20. The medical device hub of claim 15, further comprising a charge indicator.
